(19) United States
(12) Patent Application Publication
(10) Pub. No.: US 2025/0092111 A1
(43) Pub. Date: Mar. 20, 2025

(54) POTENT AND STABLE POLYPEPTIDE ANALOGUES VIA SERINE/THREONINE LIGATION

(71) Applicant: UNIVERSITY OF WASHINGTON, Seattle, WA (US)

(72) Inventors: David BAKER, Seattle, WA (US); Timothy CRAVEN, Seattle, WA (US); Paul LEVINE, Seattle, WA (US)

(21) Appl. No.: 18/727,879

(22) PCT Filed: Jan. 12, 2023
(86) PCT No.: PCT/US2023/060522

§ 371 Date: Jul. 10, 2024

Related U.S. Application Data
(60) Provisional application No. 63/299,884, filed on Jan. 14, 2022.

(51) Int. Cl. C07K 14/605; A61K 38/00; A61K 47/54

(52) U.S. Cl. CPC C07K 14/605; A61K 47/542; A61K 38/00

(57) ABSTRACT

Modified polypeptides comprising an amino acid sequence having a lysine or derivative thereof functionalized at N⁶ with a seryl or threonyl group are disclosed, as are bioconjugates comprising the modified polypeptides, and methods for making the bioconjugates.

### CROSS REFERENCE

This application claims priority to U.S. Provisional Patent Application Ser. No. 63/299,884 filed Jan. 14, 2022, incorporated by reference herein in its entirety

### SEQUENCE LISTING STATEMENT

A computer readable form of the Sequence Listing is filed with this application by electronic submission and is incorporated into this application by reference in its entirety. The Sequence Listing is contained in the file created on Dec. 23, 2022 having the file name “21-1624-WO.xml” and is 7 kb in size.

### BACKGROUND

Peptide therapeutics are rapidly becoming approved for clinical use due to their ability to engage their targets with high affinity and specificity. Although a number of peptide drugs have been approved by the U.S. Food and Drug Administration (FDA), such as teriparatide and 20 angiotensin II, they often suffer from poor pharmacokinetic profiles in vivo that likely arise from proteolytic degradation by endogenous enzymes. For example, GLP-1(7-37) has a half-life of only ˜2 min due to degradation by dipeptidyl peptidase (DPP-4) cleavage at N-terminal alanine 8. Current strategies aimed at addressing issues with stability are inadequate, as they often compromise potency at the expense of stability.

### SUMMARY

In one aspect, the disclosure provides modified polypeptides comprising an amino acid sequence having a lysine or derivative thereof functionalized at M with a seryl or threonyl group. In various embodiments, the modified polypeptides comprise the N⁶-L-seryl-functionalized lysine

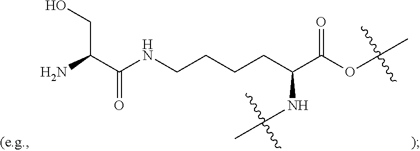 [SMILES: *N[C@@H](CCCCNC(=O)[C@@H](N)CO)C(=O)O*]

the N⁶-L-seryl-functionalized lysine derivative

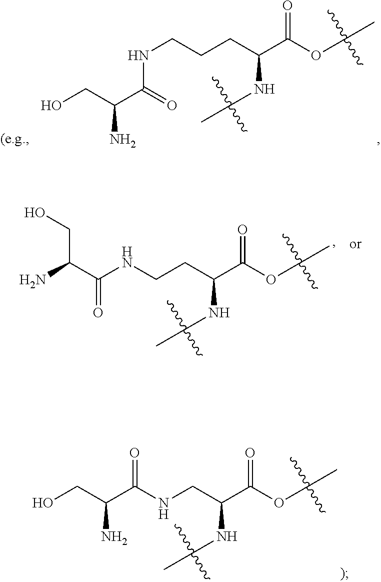 [SMILES: *N[C@@H](CCCNC(=O)[C@@H](N)CO)C(=O)O* ; *N[C@@H](CCNC(=O)[C@@H](N)CO)C(=O)O* ; *N[C@@H](CNC(=O)[C@@H](N)CO)C(=O)O*]

the N⁶-L-threonyl-functionalized lysine

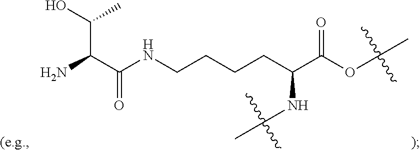 [SMILES: *N[C@@H](CCCCNC(=O)[C@@H](N)[C@@H](C)O)C(=O)O*]

or the N⁶-L-threonyl-functionalized lysine derivative

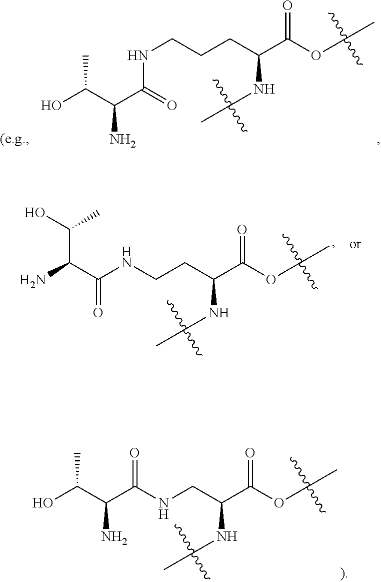 [SMILES: *N[C@@H](CCCNC(=O)[C@@H](N)[C@@H](C)O)C(=O)O* ; *N[C@@H](CCNC(=O)[C@@H](N)[C@@H](C)O)C(=O)O* ; *N[C@@H](CNC(=O)[C@@H](N)[C@@H](C)O)C(=O)O*]

In certain embodiments, the polypeptide comprises an amino acid sequence that is at least 90%, or 92%, or 94%, or 95% or more, identical to glucagon-like peptide 1 fragment 7-37 (GLP-1(7-37)), parathyroid hormone fragment 1-34 (PTH(1-34)), or peptide YY 3-36 (PYY(3-36)). In other embodiments, the polypeptide comprises a peptide therapeutic selected from the group consisting of therapeutic peptides listed in Table 1, modified to comprise a lysine or a derivative thereof functionalized at M with a seryl or threonyl group. In various embodiments, the N⁶-functionalized lysine or derivative thereof is incorporated into the amino acid sequence in place of a native lysine, or in place of a native amino acid other than lysine, including but not limited to serine.

In another embodiment, the disclosure provides bioconjugates comprising a modified polypeptide of any embodiment or combination of embodiments herein and a functional moiety, wherein the functional moiety is conjugated to the modified polypeptide at the seryl or threonyl group on the N⁶-functionalized lysine or derivative thereof. In various embodiments, the functional moiety may comprise a polyethylene glycol molecule, a lipid molecule, a fluorescent molecule, a chemiluminescent molecule, a phosphorescent molecule, a radioisotope, an enzyme, an enzyme substrate, an affinity molecule, a ligand, an antigen, a hapten, an antibody, an antibody fragment, a peptide, a peptidomimetic, a protein, a chromogenic substrate, a contrast agent, an MRI contrast agent, a PET label, a phosphorescent label, or a combination thereof. I certain embodiments, the functional moiety my comprise:

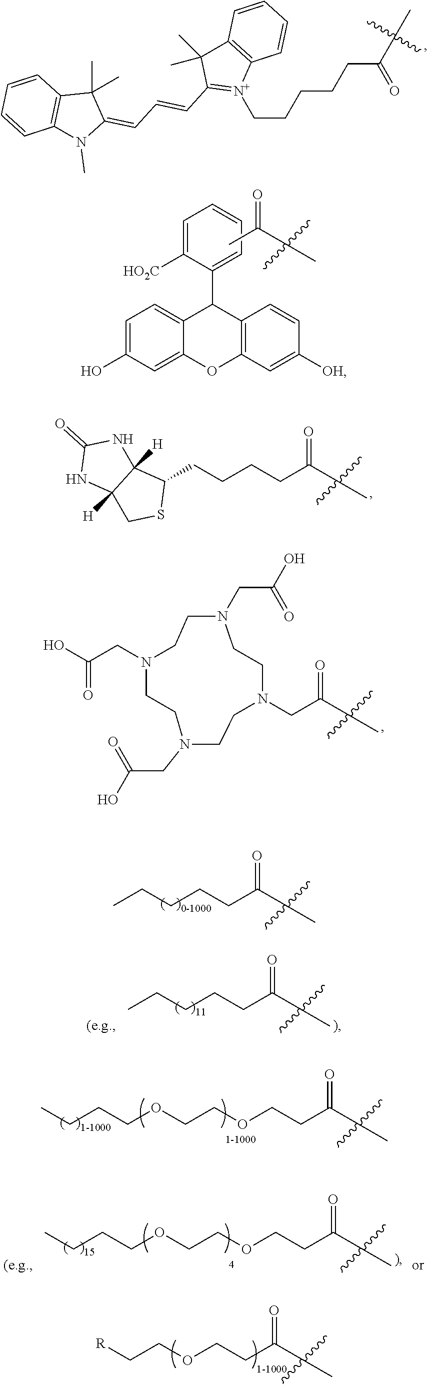

where R═NH², OH, or OCH³

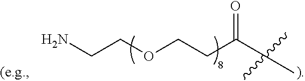

The disclosure also provides pharmaceutical compositions comprising one or more polypeptide and/or bioconjugate according to any embodiment or combination of embodiments herein and a pharmaceutically acceptable carrier, solvent, adjuvant, and/or diluent.

In another aspect, the disclosure provides methods of preparing a bioconjugate, the method comprising: contacting a modified polypeptide comprising an amino acid sequence having a lysine or derivative thereof functionalized at M⁶ with a seryl or threonyl group with a functionalized salicylaldehyde ester, wherein the salicylaldehyde ester reacts with the seryl or threonyl group on the N⁶-functionalized lysine or derivative thereof to obtain the bioconjugate. In one embodiment, the functionalized salicylaldehyde ester is of formula:

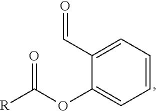

wherein R is moiety that comprises a polyethylene glycol molecule, a lipid molecule, a fluorescent molecule, a chemiluminescent molecule, a phosphorescent molecule, a radioisotope, an enzyme, an enzyme substrate, an affinity molecule, a ligand, an antigen, a hapten, an antibody, an antibody fragment, a peptide, a peptidomimetic, a protein, a chromogenic substrate, a contrast agent, an MRI contrast agent, a PET label, a phosphorescent label, or a combination thereof; or a fluorescent molecule, biotin, a polyethylene glycol molecule, a lipid molecule, or a combination thereof).

In another embodiment, the functionalized salicylaldehyde ester is of formula:

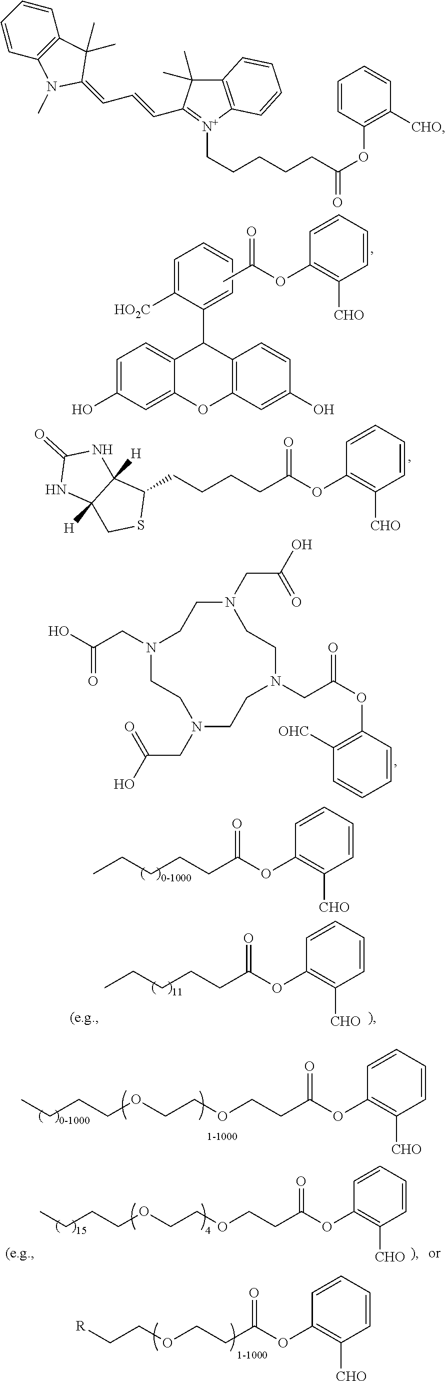

where R═NH₂, OH, or OCH₃

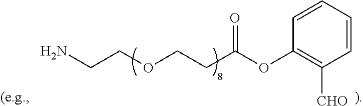

In various embodiments, the method further comprises introducing a N⁶-seryl-lysine or derivative thereof or N⁶-threonyl-lysine or derivative thereof during the modified polypeptide synthesis. In other embodiments, the modified polypeptide comprises a N-terminal serine or threonine, and the method further comprises protecting the N-terminal serine or threonine with a protecting group prior to contacting with the functionalized salicylaldehyde ester.

### DESCRIPTION OF THE FIGURES

FIG. 1. Chemical ligation at serine. (Top) Previous efforts have mainly used serine/threonine ligation (STL) in protein chemical synthesis, semi-synthesis, or peptide cyclization. (Bottom) Cartoon of methods using a non-canonical amino acid containing the 1-amino-2-hydroxy functionality required for ligation to internally generate site-specific modifications.

FIG. 2. Design of GLP-1 peptide analogues. Primary sequence of GLP-1 (SEQ ID NO: 1) and Semaglutide (SEQ ID NO: 4). Peptides G1 (SEQ ID NO: 5) and G2 (SEQ ID NO: 6), synthesized here via STL, contain a C₁₈-PEG₄ modification at position 26 in combination with an Aib residue at position 8 or just a C₁₈-PEG₄ modification at position 18.

FIG. 3. Lipidation does not impact cellular activity, stabilizes GLP-1 from proteolysis, and improves glucose clearance in vivo. (a) Lipid alone or with Aib substitution does not affect the EC₅₀ of cAMP production when compared to unmodified GLP-1 (n=3).. (Models of full length (b) GLP-1R-Semaglutide, (c) GLP-1R-G1, and (d) GLP-1R-G2 complexes.

FIG. 4. Scheme 1. Site-specific modification of an unprotected model peptide via STL. All reactions were conducted at a concentration of 10 mM in pyridine/acetic acid (1:1 v/v) followed by cleavage using TFA/H₂O/i-Pr₃SiH (94/5/1, v/v/v).

### DETAILED DISCLOSURE

All references cited are herein incorporated by reference in their entirety.

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), valine (Val; V), and alpha-aminoisobutyric acid (AIB, B).

All embodiments of any aspect of the invention can be used in combination, unless the context clearly dictates otherwise.

In a first aspect, the disclosure provides modified polypeptides comprising an amino acid sequence having a lysine or a derivative thereof functionalized at N⁶ with a seryl or threonyl group. As shown in the examples that follow the inventors demonstrated that such modified polypeptides can readily be internally and site-specifically modified for various applications in chemical biology, including the generation of potent and stable variants exemplified in non-limiting fashion by work with semaglutide.

The polypeptide may comprise a functionalized lysine or a functionalized lysine derivative. Any lysine derivative that can be functionalized at N⁶ with a seryl or threonyl group may be used. In various embodiments, the derivatives of lysine include, but are not limited to,

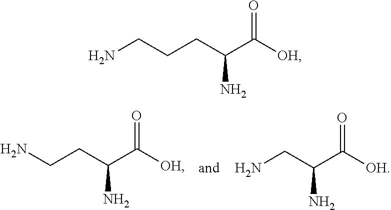 [SMILES: NCCC[C@H](N)C(=O)O ; NCC[C@H](N)C(=O)O ; NC[C@H](N)C(=O)O]

For example, in certain embodiments, the modified polypeptide comprises the N⁶-L-seryl-functionalized lysine

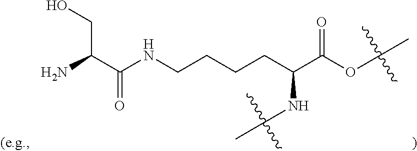 [SMILES: *N[C@@H](CCCCNC(=O)[C@@H](N)CO)C(=O)O*]

or the N⁶-L-seryl-functionalized lysine derivative

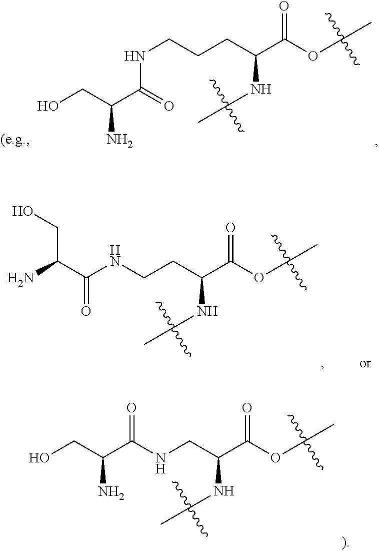 [SMILES: *N[C@@H](CCCNC(=O)[C@@H](N)CO)C(=O)O* ; *N[C@@H](CCNC(=O)[C@@H](N)CO)C(=O)O* ; *N[C@@H](CNC(=O)[C@@H](N)CO)C(=O)O*]

In certain embodiments, the modified polypeptide comprises the N⁶-L-threonyl-functionalized lysine

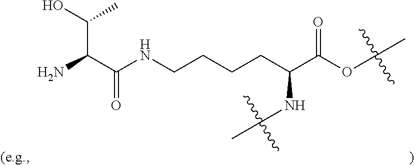 [SMILES: *N[C@@H](CCCCNC(=O)[C@@H](N)[C@@H](C)O)C(=O)O*]

or the N⁶-L-threonyl-functionalized lysine derivative

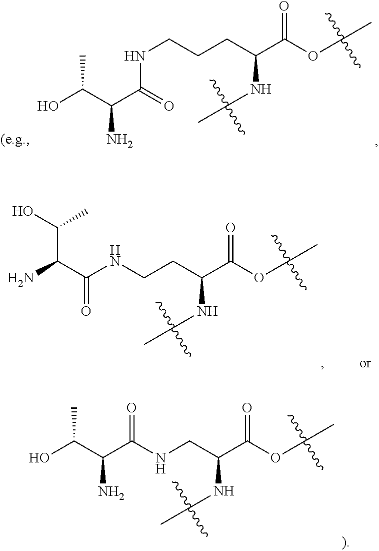 [SMILES: *N[C@@H](CCCNC(=O)[C@@H](N)[C@@H](C)O)C(=O)O* ; *N[C@@H](CCNC(=O)[C@@H](N)[C@@H](C)O)C(=O)O* ; *N[C@@H](CNC(=O)[C@@H](N)[C@@H](C)O)C(=O)O*]

In some embodiments, the N⁶-functionalized lysine or derivative thereof comprises a protecting group. For example, the N⁶-functionalized lysine may further comprise a Boc-protecting group or t-butyl protecting group, or a combination thereof.

Suitable N⁶-functionalized lysines may be prepared as known in the art. For example, the preparation of certain functionalized lysine peptides, such as serine-lysine conjugates, can be found in C. H. P. Cheung, J. Xu, C. L. Lee, Y. Zhang, R Wei, D. Bierer, X. Hunag, and X. Li, Chem. Sci., 2021, 12, 7091, which is incorporated herein by reference in its entirety.

As will be understood by those of skill in the art, the N⁶-functionalized lysine or derivative thereof is incorporated into the amino acid sequence in place of a native lysine or in place of another native amino acid. The N⁶-functionalized lysine or derivative thereof may be incorporated in place of any amino acid in the unmodified polypeptide. In one embodiment, the M-functionalized lysine or derivative thereof is incorporated in place of a lysine residue in the unmodified polypeptide. In another embodiment, the N⁶-functionalized lysine or derivative thereof is incorporated in place of a serine residue in the unmodified polypeptide. In one embodiment, the modified polypeptide may comprise two or more N⁶-functionalized lysine or derivative thereof.

The polypeptide may be any polypeptide that could benefit from functionalization to improve stability or other polypeptide characteristics. In various non-limiting embodiments, the polypeptide may comprises a modified version of a peptide therapeutic selected from the group consisting of therapeutic peptides listed in Table 1, modified to comprise a lysine or a derivative thereof functionalized at N⁶ with a seryl or threonyl group. In some embodiments, the modified polypeptide is at least 90%, or 92%, or 94%, or 95%, or 96%, or 97%, or 98% or more, identical to the amino acid sequence of the unmodified polypeptide

TABLE 1
(adapted from creative-peptides.com/blog/list-of-marketed-peptide-drugs)

| Type | Name |
|---|---|
| Adrenocorticotropic hormone (ACTH) and its derivatives | Corticorelin |
| | Cosyntropin |
| | Seractide |
| Cholecystokinin analogs | Ceruletide |
| | Sincalide |
| Thyroid Stimulating Hormone Releasing Hormone | Taltirelin |
| | Protirelin |
| Growth hormone releasing hormone (GHRH) and analogs | Sermorelin |
| | Somatorelin |
| | Tesamorelin |
| Secretin | Secretin (human) |
| | Secretin (porcine) |
| Thymus hormone | Thymalfasin |
| | Thymopentin |
| Calcitonin | Salmon Calcitonin |
| | Elcatonin |
| | Human Calcitonin |
| Parathyroid hormone derivatives | Teriparatide |
| Oxytocin analogs and antagonists | Atosiban |
| | Carbetocin |
| | Oxytocin |
| Gonadotropin-releasing hormone (GnRH) and analogues | Buserelin |
| | Gonadorelin |
| | Goserelin |
| | Histrelin |
| | Leuprolide |
| | Nafarelin |
| | Triptorelin |
| Gonadotropin releasing hormone (GnRH) antagonist | Abarelix |
| | Cetrorelix |
| | Degarelix |
| | Ganirelix |
| Somatostatin analogs and antagonists | Depreotide |
| | Edotreotide |
| | Lanreotide |
| | Octreotide |
| | Pentetreotide |
| | Somatostatin |
| | Vapreotide |
| Vasopressin analogs | Argipressin |
| | Desmopressin |
| | Lypressin |
| | Phenypressin |
| | Terlipressin |
| Anti-HIV drugs | Enfuvirtide |
| Central Nervous System | Glatiramer |
| Peripheral nervous system | Ziconotide |
| Cardiovascular | Saralasin |
| | Bivalirudin |
| | Eptifibatide |
| | Carperitide |
| | Nesiritide |
| Angiotensin | Icatibant |
| Diabetes | Exenatide |
| | Liraglutide |
| | Lixisenatide |
| | Albiglutide |
| | Dulaglutide |
| | Pramlintide |
| Diabetes | Semaglutide |
| Others | Linasclotide |
| | Sinaspultide |
| | Pasircotide |
| | Teduglutide |
| | Peginesatide |
| | Pentagastrin |
| | Aviptadil |

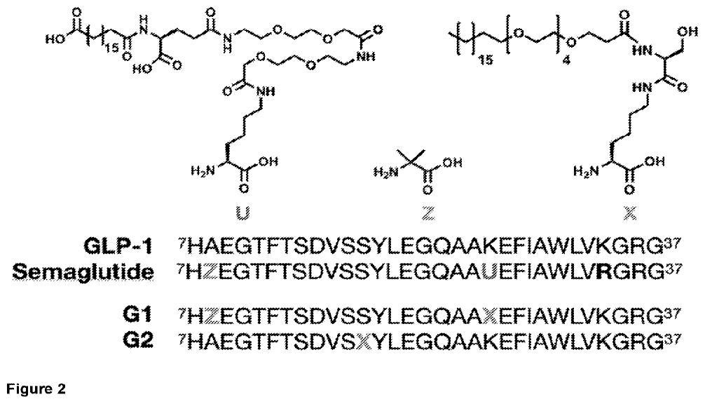

In one non-limiting embodiment, the modified polypeptide comprises an amino acid sequence that is at least 900%, or 92%, or 94%, or 95% or more, identical to glucagon-like peptide 1 fragment 7-37 (GLP-1(7-37)) (SEQ ID NO:1). The amino acid sequence of GLP-1(7-37) is shown in FIG. 2.

In another embodiment, the polypeptide comprises an amino acid sequence that is at least 90%, or 92%, or 94%, or 95% or more, identical to parathyroid hormone fragment 1-34 (PTH(1-34)) (SEQ ID NO:2).

(SEQ ID NO: 2)
SVSEIQLMHNLGKHLNSMERVEWLRKKLQDVHNF

In a further embodiment, the polypeptide comprises an amino acid sequence that is at least 85%, or 90/, or 92%, or 95% or more, identical to peptide YY 3-36 (PYY(3-36)) (SEQ ID NO:3).

(SEQ ID NO: 3)
IKPEAPGEDASPEELNRYYASLRHYLNLVTRQRY

In another aspect, the disclosure provides bioconjugates comprising a modified polypeptide of the disclosure as described herein and a functional moiety conjugated to the modified polypeptide at the seryl or threonyl group on the N⁶-functionalized lysine or derivative thereof. The modified polypeptide of the disclosure can accept any functional moiety as deemed appropriate for an intended use. In non-limiting embodiments, the functional moiety may comprise a polyethylene glycol molecule, a lipid molecule, a fluorescent molecule, a chemiluminescent molecule, a phosphorescent molecule, a radioisotope, an enzyme, an enzyme substrate, an affinity molecule, a ligand, an antigen, a hapten, an antibody, an antibody fragment, a peptide, a peptidomimetic, a protein, a chromogenic substrate, a contrast agent, an MRI contrast agent, a PET label, a phosphorescent label, or a combination thereof. In one specific embodiment, the functional moiety may comprise a fluorescent molecule, biotin, a polyethylene glycol molecule, a lipid molecule, or a combination thereof. In other embodiments, the functional moiety may comprises a moiety selected from the group consisting of.

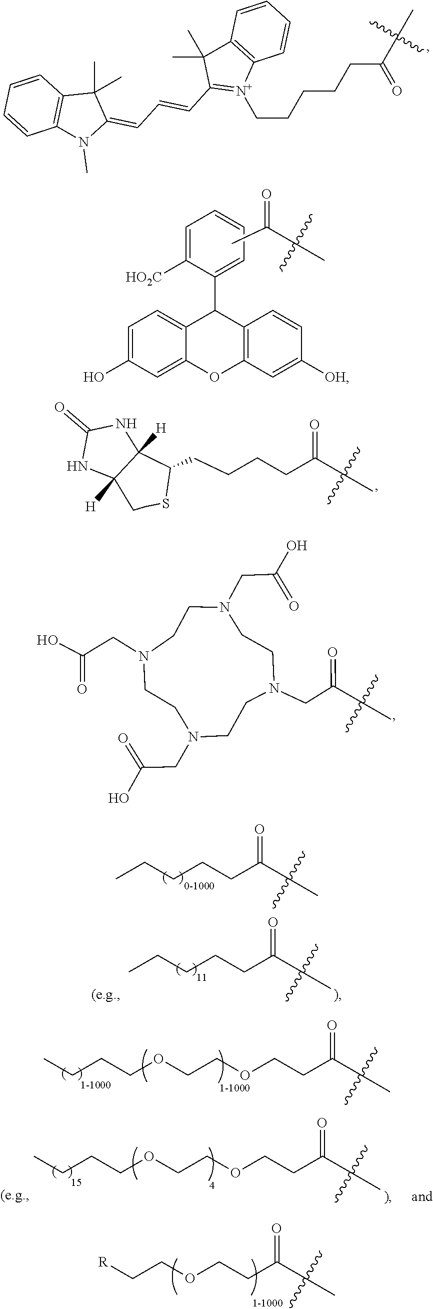

where R═NH₂, OH, or OCH₃

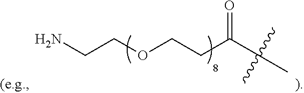

In various of these embodiments, the functional moiety increases biostability of the bioconjugate as compared to the biostability of the native (unmodified) polypeptide. As shown in the examples below, the bioconjugates of the disclosure, exemplified in non-limiting fashion by work with semaglutide, are potent and stable analogues of the unmodified polypeptide, with improved stability compared to the unmodified polypeptide.

The disclosure also provides a pharmaceutical composition comprising one or more polypeptides and/or bioconjugates according to the disclosure as described herein, wherein the modified polypeptide comprises a therapeutic protein or peptide, and a pharmaceutically acceptable carrier, solvent, adjuvant, and/or diluent. The pharmaceutical compositions of the disclosure can be used, for example, in methods for treating a subject in need of a therapy for a disorder that the polypeptide is designed to treat. The pharmaceutical composition may comprise in addition to the polypeptide or bioconjugate of the disclosure (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides and/or bioconjugates may be the sole active agent in the pharmaceutical composition, or the composition may further comprise one or more other active agents suitable for an intended use.

In another aspect, the disclosure provides methods of preparing a bioconjugate of the disclosure as described herein. Such methods include: contacting a modified polypeptide comprising an amino acid sequence having a lysine or derivative thereof functionalized at M with a seryl or threonyl group with a functionalized salicylaldehyde ester, wherein the salicylaldehyde ester reacts with the seryl or threonyl group on the N⁶-functionalized lysine or derivative thereof to obtain the bioconjugate. In embodiments wherein the modified polypeptide comprises a protecting group, the method of preparing a bioconjugate may further comprise a step of treating the modified polypeptide to remove the protecting group.

Any suitable functionalized salicylaldehyde ester may be used in the methods. In one embodiment, the functionalized salicylaldehyde ester is of formula:

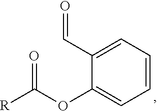

wherein R is moiety that comprises a polyethylene glycol molecule, a lipid molecule, a fluorescent molecule, a chemiluminescent molecule, a phosphorescent molecule, a radioisotope, an enzyme, an enzyme substrate, an affinity molecule, a ligand, an antigen, a hapten, an antibody, an antibody fragment, a peptide, a peptidomimetic, a protein, a chromogenic substrate, a contrast agent, an MRI contrast agent, a PET label, a phosphorescent label, or a combination thereof.

In one embodiment, R is a fluorescent molecule, biotin, a polyethylene glycol molecule, a lipid molecule, or a combination thereof. In another embodiment, the functionalized salicylaldehyde ester is of a formula selected from the group consisting of:

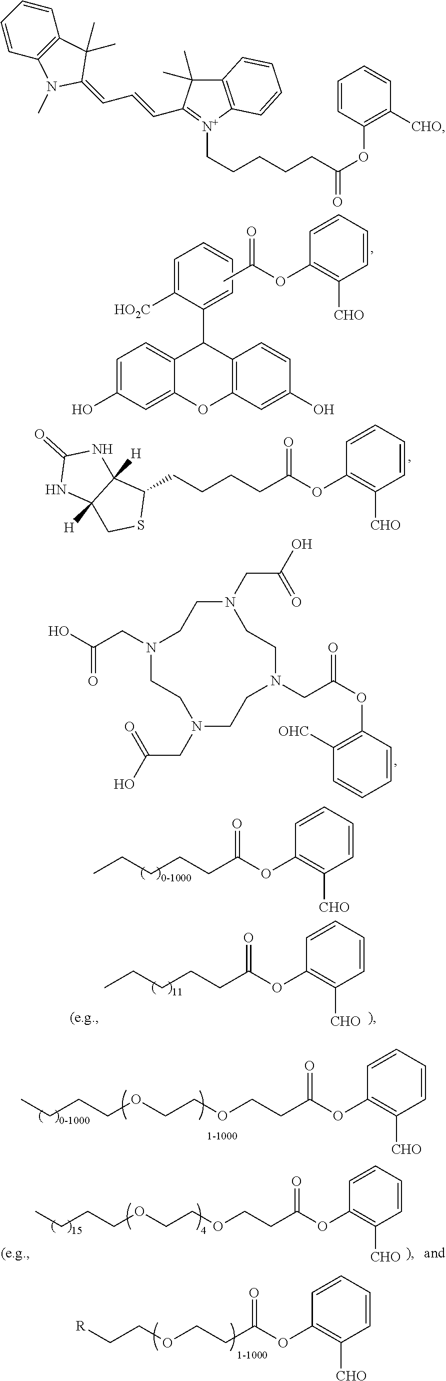

where R═NH₂, OH, or OCH₃

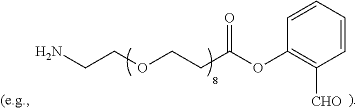

Many peptide-based therapeutics are unstable due to protease-catalyzed degradation. In a further embodiment, the R moiety increases biostability of the bioconjugate as compared to the biostability of the modified polypeptide. The modified polypeptide may be according to any embodiment or combination of embodiments described herein. Biostability may be determined by incubating the peptide in human serum and monitoring the peptide through chromatography, such as revese-phase HPLC. In particular embodiments, the bioconjugate has a halflife in human serum of at least 12 hours, for example, at least 16 hours, or at least 24 hours, or at least 30 hours.

The modified polypeptide may be contacted with the functionalized salicylaldehyde ester in any suitable solution. In one embodiment, the modified polypeptide is contacted with the functionalized salicylaldehyde ester in a solution comprises a nitrogenous base and an organic acid, such as a pyridine/acetic acid solution. The pyridine/acetic acid may be in any suitable ratio in the solution, such as in the range of 0.2:1 to 5:1 v/v pyridine:acetic acid, including but not limited to a 1:1 v/v ratio.

The modified polypeptide may contacted with the functionalized salicylaldehyde ester at any suitable temperature range. In one embodiment, the modified polypeptide is contacted with the functionalized salicylaldehyde ester at temperature in a range of about 18° C. to 27° C. for a period of time sufficient to form a N,O-benzylidene acetal intermediate. In a further embodiment, the N,O-benzylidene acetal intermediate is reacted under acidic conditions for a period of time sufficient to form the bioconjugate. Any suitable acidic conditions may be used. In one embodiment, the acidic conditions comprise use of trifluoroacetic acid.

In a further embodiment, the method further comprises introducing a N⁶-seryl-lysine or derivative thereof or N⁶-threonyl-lysine or derivative thereof during the modified polypeptide synthesis.

In another embodiment, if the modified polypeptide comprises an N-terminal serine or threonine, the method may further comprise protecting the N-terminal serine or threonine with a protecting group prior to contacting with the functionalized salicylaldehyde ester. Any suitable protecting group may be used, including but not limited to allyl serine or N-terminal acetylation.

During any of the processes for preparation of the subject compounds, polypeptides, or bioconjugats, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

### EXAMPLES

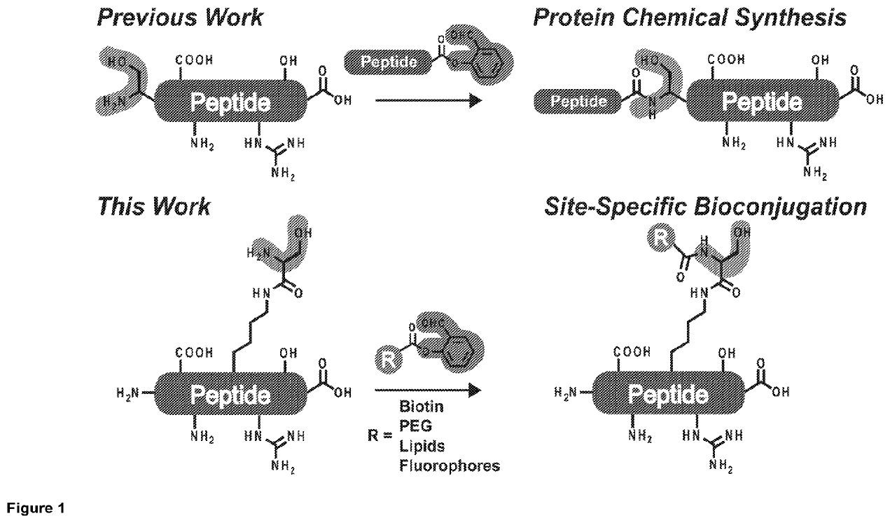

Peptide and protein bioconjugation permit site-specific introduction of a variety of functional groups for different applications, including proteomics and high-resolution imaging. One such method is Ser/Thr ligation (STL), which is a chemoselective reaction that occurs between a C-terminal salicylaldehyde ester and an N-terminal fragment containing a serine or threonine residue that undergoes reversible imine formation via aldehyde capture. Following an acyl shift, a stable N,O-benzylidene acetal intermediate can be cleaved with acid to liberate a native serine/threonine linkage at the ligation site. N,O-benzylidene acetal intermediate only forms in the presence of a 1-amino-2-hydroxy function, such as present for N-terminal serine or threonine. Here we utilize a non-canonical amino acid containing the 1-amino-2-hydroxy functionality to internally and site-specifically modify peptides for various applications in chemical biology, including the generation of potent and stable variants of GLP-1(7-37) (FIG. 1).

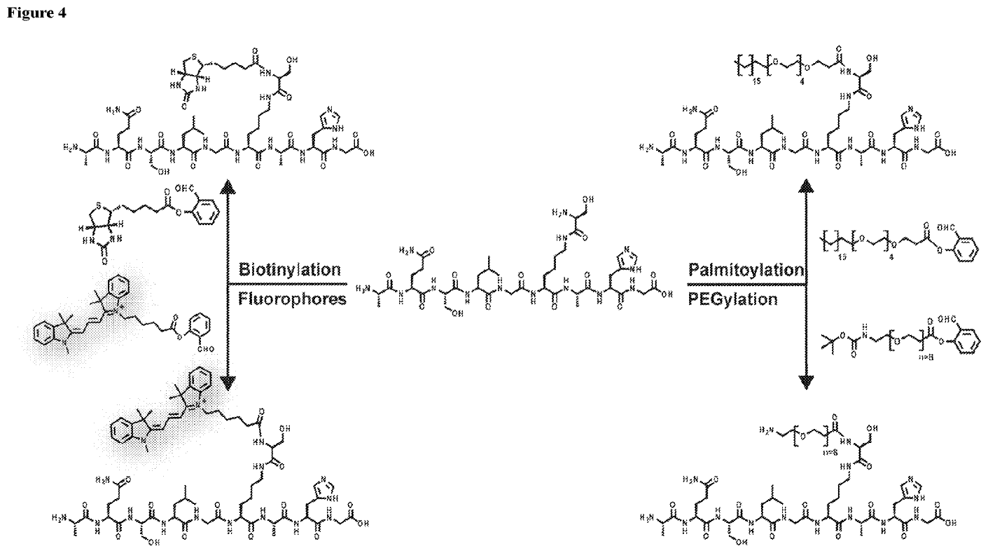

To assess the scope and generality of this approach, we first synthesized biotin, cyanine-3, a palmitic acid analogue, and monodisperse poly-ethylene glycol salicylaldehyde esters from commercially available starting materials in one step. All probes were then site-specifically installed onto a model peptide containing the 1-amino-2-hydroxy non-canonical amino acid (Scheme 1; FIG. 4). Following bioconjugation, which was monitored by high-performance liquid chromatography, all products were characterized by electrospray ionization mass spectrometry.

Next, we explored how we could harness this bioconjugation strategy to enhance the stability of peptide therapeutics. The most common strategy to extend the half-life of peptide and protein therapeutics is PEGylation and lipidation. Two GLP-1(7-37) drugs, Semaglutide and Liraglutide, are lipidated and currently used to manage blood glucose for the treatment of type 2 diabetes. Both PEGylation and lipidation provide protection from protease-catalyzed degradation. Additionally, lipidation promotes binding to circulating human albumin, which releases drugs at a slow, constant rate.

With this in mind, we used STL to synthesize two analogues of GLP-1 that resemble Semaglutide (FIG. 2), which contains a hybrid PEG and fatty acid side-chain. The first peptide (G1) was modified at lysine 26, the same position as Semaglutide. As this site is further away from the DPP-4 cleavage site at alanine 8, we also included 2-aminoisobutyric acid (AIB) in place of alanine 8, similar to Semaglutide, to provide additional stability. The main difference between Semaglutide and G1, aside from the subtle side-chain modification, is that G1 maintains the native lysine 34 as conjugation is site-specific with STL. The second peptide (G2) was modified at serine 18. In this particular case, we chose to omit Aib at alanine 8 since the lipid is closer to the N-terminus and likely to shield proteolysis better. Characterization data for the two analogues is summarized in Table 2.

TABLE 2

| Peptide | Purity | Mass Calc/Obs |
|---|---|---|
| G1 | ~95% | 3955/3955 |
| G2 | ~95% | 3983/3983 |

Many biochemical and structural studies have demonstrated that an extended amphipathic α-helix within GLP-1 is responsible for high affinity binding interactions with the extracellular domain of the GLP receptor. To assess how these modifications might disrupt secondary structure, we used circular dichroism (CD) spectroscopy to observe any changes relative to GLP-1. Relative to GLP-1, which displays a characteristic helical fold, both G1 and G2 also show helical structure, however less than native GLP-1. This data is consistent with the lipid modification found on Semaglutide and this loss in structure seems to be induced by the lipid modification.

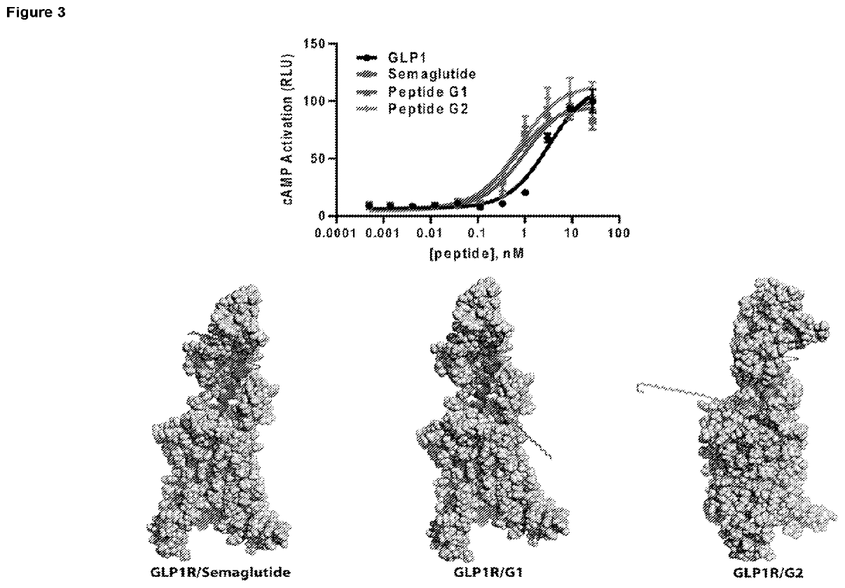

Endogenous binding of GLP-1 to the GLP-1R results in an intracellular rearrangement that allows recruitment of G-protein, subsequently stimulating the production of cyclic AMP (cAMP) from ATP and leading to glucose-stimulated insulin secretion.¹⁹ To assess the ability of the lipid modified GLP-1 analogs G1 and G2 to activate human GLP-1R, cAMP accumulation was measured in CHO-K1 cells overexpressing the human GLP-1R. Cells were initially treated with native GLP-1 and Semaglutide as reference agonists, which exhibited EC₅₀'s of 3.3±0.6 nM and 0.60±0.2 nM (mean±s.e.m., n=3), respectively (FIG. 3a). In comparison, both G1 and G2 performed better than unmodified peptide and were roughly equipotent to Semaglutide, with EC₅₀ 's of 0.97±0.2 nM and 0.73±0.2 nM (mean±s.e.m., n=3), respectively. This data suggests that neither lipid modification on Lys26 or Ser18 significantly perturbs endogenous function. To complement our in vitro pharmacological profiling of the lipid modified GLP-1 analogues G1 and G2, the stability of the compounds relative to native GLP-1 and Semaglutide in human serum was compared using reverse-phase high-performance liquid chromatography (RP-HPLC) (data not shown). As expected, native GLP-1 displayed a relatively short half-life in this assay, t_{1/2}=˜3.5 hr, as the N-terminal Ala8 residue is readily cleaved. In contrast, Semaglutide showed almost no sign of degradation up to 48 h, as its stability is significantly enhanced by the addition of Aib at Ala8 and the lipid modification. These half-lives are consistent with previous reports. Relative to native GLP-1, G1 displayed a significantly improved stability profile, t_{1/2}=˜40 hr. Although G1 contains Aib substituted at Ala8 to prevent cleavage by DPP4, this data suggests that other proteases present in human serum can degrade G1 at other sites. Lastly, G2 proved to be very stable, with a more than a 14-fold increase in stability relative to native GLP-1, very comparable to Semaglutide.

Given the promising activation and stability data, the peptides were tested in vivo using a standard glucose tolerance test (GTT). Both G1 and G2 displayed improved glucose disposal efficiency compared to unmodified GLP-1 (data not shown), consistent with their in vitro data. These data highlight the ability of lipidation to significantly increase stability without compromising potency, thus resulting in improved in vivo activity in a mouse model of glucose disposal.

In order to gain molecular insight into how G1 and G2 interact with the GLP-1R, we performed computational modeling of the corresponding ligand-receptor complexes, as described in the experimental methods. The GLP-1R peptide binding models were based on the recently published Cryo-EM structure of GLP-1R in complex with unmodified GLP-1 peptide. The generated models of Semaglutide, G1, and G2 in their complexes with the GLP-1R suggest that lipidation at either serine 18 or lysine 26 are solvent exposed and likely not interfering with any critical contacts responsible for binding or activation (FIG. 3b-d). Additionally, the lipid modification of G1 is closer to the N-terminus of the peptide, possibly indicating there is an exposed degradation site between the C-terminal Aib residue and the lipid modification.

In conclusion, we introduce a robust site-specific bioconjugation strategy that relies on ‘serine ligation’. A multitude of salicyaldehyde ester probes can be easily synthesized in one step from commercially available carboxylic acids to generate peptides with stable linkages for various applications in chemical biology and medicine. Unlike the chemoselective chemistry that is currently used to install the lipid functional group on Semaglutide, which entails mutation of any other native lysine residue in the native sequence, our site-specific strategy does not require this. N-terminal serine or threonine residues in peptides may compete for modification; however this can be avoided by utilizing a simple protecting group strategy, such as allyl serine or N-terminal acetylation. We applied this technology to produce potent and stable GLP-1 analogues, outfitted with a hybrid PEG and fatty acid side-chain that resemble the widely used diabetes drug Semaglutide. Both compounds were equipotent to Semaglutide in their ability to activate GLP-1R, displayed significantly improved stability profiles in human serum relative to native GLP-1, and outperformed GLP-1 in vivo.

In certain embodiments, STL bioconjugation strategy may be used to create potent and stable analogues of other GPCRs, such as PTH(1-34). Additionally, in certain embodiments, the STL bioconjugation is combined with amber stop codon technology to scale production of the modified peptide of the disclosure by eliminating solid phase peptide synthesis. This approach demonstrates the potential for creating peptides for an assortment of applications, with a particular emphasis on therapeutic peptides.

### Abbreviations

GPCRs, G protein-coupled receptors; DPP-4, dipeptidyl peptidase; STL, Ser/Thr ligation; Aib, 2-aminoisobutyric acid; GTT, glucose tolerance test.

Materials: Biotin was purchased from Sigma Aldrich. Cyanine3 acid was purchased from lumiprobe. C18-PEG4-COOH and t-boc-N-amido-sPEG8-acid were purchased from creative PEGworks and Quanta Biodesign, respectively. Semaglutide acetate was purchased from Bachem. Synthesis of the Lys-Ser dipeptide was conducted by WuXi AppTec as previously reported (C. H. P. Cheung, J. Xu, C. L. Lee, Y. Zhang, R. Wei, D. Bierer, X. Hunag, and X. Li, Chem. Sci., 2021, 12, 7091). All other reagents were obtained from commercial sources and used without additional purification. All aqueous solutions were prepared using ultrapure laboratory grade water (deionized, filtered, and sterilized) obtained from an in-house ELGA water purification system. Reverse-phase high-performance liquid chromatography (RP-HPLC) was performed using an Agilent Technologies 1260 Series HPLC instrument with a diode array detector. For analytical analysis, a C18 reversed-phase HPLC column was used (Higgins). Samples were eluted with a 5-95% acetonitrile/water gradient (0.1% TFA) in 45 minutes with a flow rate of 1 mL/min and monitored at 214 nm. For purifications, semi-preparative C18 reversed-phase HPLC columns were used (Higgins). Samples were eluted with a 5-65% or 25-95% acetonitrile/water gradient (0.1% TFA) in 35 minutes with a flow rate of 5.0 ml/min, and monitored at 220 nm. Mass spectra were acquired on an Agilent LC-TOF or Thermo ESI direct inject mass spec.

Peptide Synthesis: All peptides were synthesized using standard Fmoc solid-phase chemistry on either 2-Chlorotrityl ProTide (CEM, 0.45 mmol/g) or Rink amide ChemMatrix (PCAS BioMatrix, 0.45 mmol/g) resin using a Liberty Blue peptide synthesizer from CEM. Couplings were performed using DIC (5 equiv, Novabiochem) and Oxyma (10 equiv, Sigma) in DMF followed by Fmoc deprotection with 20% piperidine. All peptides were then cleaved (95:2.5:2.5 TFA/H₂O/triisopropylsilane) for 3.5 h at room temperature, precipitated out of cold ether, and purified by reverse-phase HPLC using preparative chromatography. All peptides were characterized by mass analysis using ESI-MS, and the sequence purity was assessed by analytical HPLC.

Circular Dichroism Spectroscopy: Circular dichroism spectra were recorded on a Jasco J-1500 CD spectrometer. Peptides were freshly diluted to a final concentration of 50 μM in 10 mM phosphate buffer (pH 7.4) prior to sample measurement. Spectra were recorded from 250 to 190 nm with a 0.1 nm data pitch, a 50 nm min-1 scanning speed, a 4 sec data integration time, a 1 nm bandwidth, and a 1 mm path length with 3 accumulations, at 25° C. All data were background subtracted from a sample containing only phosphate buffer.

GLP-1 cAMP Accumulation Assay: GLP-1 receptor activation was measured using the cAMP Hunter eXpress assay kit (Eurofins DiscoverX Corporation, #95-0062E2CP2M) in CHO-K1 cells overexpressing the human GLP1R. All reagents were from the assay kit unless stated otherwise. Everything was performed according to the manufacturer's instructions. All data were analyzed using Prism 8.3.0 (GraphPad Software Inc., San Diego, CA)

### Synthesis of Salicylaldehyde Esters and Ligation Reaction Conditions

Synthesis of Salicylaldehyde Esters: Biotin, Cyanine3, C18-PEG4-COOH, and t-boc-N-amido-sPEG8-acid were added to salicylaldehyde (1.1 eq.), DIC (1.2 eq), and DMAP (0.1 eq) in dry DCM (2-4 mL). The reaction(s) were allowed to proceed overnight, resulting in salicylaldehyde esters that were purified by semi-preparative HPLC.

General Procedure for Ligation to Model Peptide: Salicylaldehyde esters (1 equiv.) and the peptide fragment (1.0 equiv.) were dissolved in pyridine/acetic acid (1:1 v/v) to a final concentration of ˜0.025-0.05 M. The reaction(s) were stirred at room temperature and monitored using LC/MS. Following completion of the reaction, the solvent was removed by lyophilization and the intermediate product was treated with TFA/H2O/i-Pr3SiH (94/5/1, v/v/v) for 10 min (2 hours for Peg with boc) to give the product containing a native amide bond at the ligation site.

General Procedure for Ligation to GLP-1 (7-37): GLP-1 (7-37) (10 mg) was dissolved in pyridine/acetic acid (1:1 v/v) to a final concentration of ˜10 mM and corresponding salicylaldehyde ester (1 equiv.) was added. The reaction was stirred at room temperature and monitored using and HPLC. Following completion of the reaction, the solvent was removed by lyophilization and the intermediate was treated with TFA/H2O/i-Pr₃SiH (94/5/1, v/v/v) for 15 min, and 2 hr for Boc protected PEG, to give the product containing a native amide bond at the ligation site.

### REFERENCES

(1) Muttenthaller, M.; King, G. F.; Adams, D. J.; Alewood, P. F. Trends in Peptide Discovery. Nat. Rev. Drug Discov. 2021, 20, 309-325.
(2) Trujillo, J. M.; Nuffer, W.; Ellis, S. L. GLP-1 Receptor Agonists: a Review of Head-to-Head Clinical Studies. Ther. Adv. Endocrinol. Metab. 2015, 6 (1), 19-28.
(3) Al-Merani, A. S.; Brooks, P. D.; Chapman, J. B.; Munday, A. K. The Half-Lives of Angiotensin II, Angiotensin II-Amide, Angiotensin III, Sar1-Ala8-Angiotensin 11 and Renin in the Circulatory System of the Rat. J. Physiol. 1978, 278, 471-490.
(4) Lovshin, J. A.; Drucker, D. J. Incretin-Based Therapies for Type 2 Diabetes Mellitus. Nat. Rev. Endocrinol. 2009, 5 (5), 262-269.
(5) Green, B. D.; Mooney, M. H.; Gault, V. A.; Irwin, N.; Bailey, C. J.; Harriott, P.; Greer, B.; O'Harte, F. P. M.; Flatt, P. R. N-Terminal His(7)-Modification of Glucagon-Like Peptide-1(7-36) Amide Generates Dipeptidyl Peptidase IV-Stable Analogues with Potent Antihyperglycaemic Activity. J. Endocrinol. 2004, 180 (3), 379-388.
(6) Chen, X.; Mietlicki-Baase, E. G.; Barrett. T. M.; McGrath, L. E.; Koch-Laskowski, K.; Ferrie, J. J.; Hayes, M. R.; Petersson, E. J. Thioamide Substitution Selectively Modulates Proteolysis and Receptor Activity of Therapeutic Peptide Hormones. J. Am. Chem. Soc. 2017, 139 (46), 16688-16695.
(7) Lin, S., Yang, X., Jia, S., Weeks, A. M., Hornsby, M., Lee, P. S., Nichiporuk, R. V., Iavarone, A. T., Wells, J. A., Toste, F. D., and Chang, C. J. Redox-Based Reagents for Chemoselective Methionine Bioconjugation. Science. 2017, 355, 597-602.
(8) Gupta, A.; Rivera-Molina, F.; Xi, Z.; Toomre, D.; Schepartz, A. Endosome Motility Defects Revealed at Super-Resolution in Live Cells Using HIDE Probes. Nat. Chem. Biol. 2020, 16, 408-414.
(9) Zhang, Y.; Xu, C.; Lam, H. Y.; Lee, C. L.; Li, X. Protein Chemical Synthesis by Serine and Threonine Ligation. Proc. Natl. Acad. Sci. U.S.A. 2013, 110 (37), 6657-6662.
(10) Levine, P. M.; Craven, T. W.; Bonneau, R; Kirshenbaum, K. emi-Synthesis of Peptoid-Protein Hybrids by Chemical Ligation at Serine. Org. Lett. 2014, 16 (2), 512-515.
(11) Knudsen, L. B., Nielsen, P. F.; Huusfeldt, P. O.; Johansen, N. L.; Madsen, K.; Pedersen, F. Z.; Thegersen, H.; Wilken, M.; Agerso, H. Potent Derivatives of Glucagon-Like Peptide-1 with Pharmacokinetic Properties Suitable for Once Daily Administration. J. Med. Chem. 2000, 43 (9), 1664-1669.
(12) Jensen, L.; Helleberg, H.; Roffel, A.; Van Lier J. J.; Bjornsdottir, I.; Pedersen, P. J.; Rowe, E.; Derving Karsbol, J.; Pedersen, M. L.; Absorption, Metabolism and Excretion of the GLP-1 Analogue Semaglutide in Humans and Nonclinical Species. Eur. J. Pharm. Si. 2017, 104, 31-41.
(13) Menacho-Melgar, R; Decker, J. S.; Hennigan, J. N.; Lynch, M. D.; A Review of Lipidation in the Development of Advanced Protein and Peptide Therapeutics. J. Control. Release. 2019, 10 (295), 1-12.
(14) Spector, A. A. Fatty Acid Binding to Plasma Albumin. J. Lipi. Res. 1975, 16 (3), 165-179.
(15) Zhang, Y.; Sun, B., Feng, D.; Hu, H., Chu, M.; Qu, Q.; Tarrasch, J. T.; Li, S.; Sun Kobilka,
T.; Kobilka, B. K.; Skiniotis, G. Cryo-EM Structure of the Activated GLP-1 Receptor in Complex with a G Protein. Nature. 2017, 546 (7657), 248-253.
(16) Levine, P. M., Balana, A. T.; Sturchler, E.; Koole, C.; Noda, H.; Zarzycka, B.; Daley, E. J.; Truong, T. T.; Katritch, V.; Gardella, T. J.; Wootten, D.; Sexton, P. M.; McDonald, P.; Pratt, M. R. O-G1cNAc Engineering of GPCR Peptide-Agonists Improves Their Stability and In Vivo Activity. J. Am. Chem. Soc. 2019, 141, 14210-14219.
(17) Adelhorst, K.; Hedegaard, B. B.; Knudsen, L. B., Kirk, O. Structure Activity Studies of Glucagon-like Peptide-1. J. Biol. Chem. 1994, 269 (9), 6275-6278.
(18) Venanzi, M.; Savioli, M.; Cimino, R.; Gatto, E.; Palleschi, A.; Ripani, G.; Cicero, D.; Placidi, E.; Orvieto, F.; Bianchi, E. A Spectroscopic and Molecular Dynamics Study on the Aggregation Process of a Long-Lasting Lipidated Therapeutic Peptide: The Case of Semaglutide. Soft Matter. 2020, 16, 10122-10131.
(19) Zhang, R.; Xie, X. Tools for GPCR Drug Discovery. Acta Pharmacol. Sin. 2012, 33 (3), 372-384.
(20) Fraher, L. J.; Klein, K.; Marier, R; Freeman, D.; Hendy, G. N.; Goltzman, D.; Hodsman, A. B. Comparison of the Pharmacokinetics of Parenteral Parathyroid Hormone-(1-34) [PTH-(1-34)] and PTH-Related Peptide-(1-34) in Healthy Young Humans. J. Clin. Endocrinol. Metab. 1995, 80 (1), 60-64.

What is claimed is:

1. A modified polypeptide comprising an amino acid sequence having a lysine or derivative thereof functionalized at N⁶ with a seryl or threonyl group.

2. The modified polypeptide of claim 1, comprising the N⁶-L-seryl-functionalized lysine 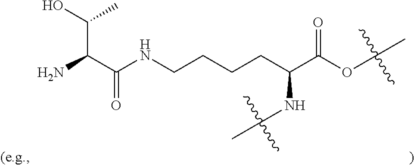 [SMILES: *N[C@@H](CCCCNC(=O)[C@@H](N)[C@@H](C)O)C(=O)O*]
or the N⁶-L-seryl-functionalized lysine derivative

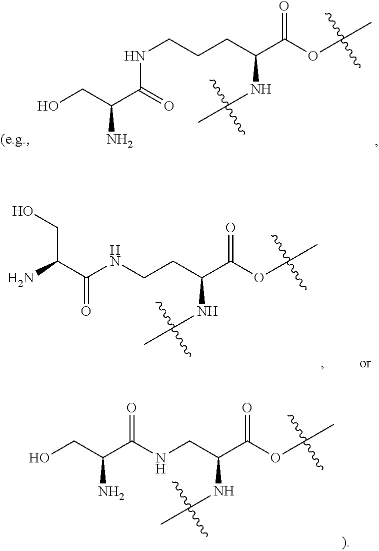 [SMILES: *N[C@@H](CCCNC(=O)[C@@H](N)CO)C(=O)O* ; *N[C@@H](CCNC(=O)[C@@H](N)CO)C(=O)O* ; *N[C@@H](CNC(=O)[C@@H](N)CO)C(=O)O*]

3. The modified polypeptide of claim 1, comprising the N⁶-L-threonyl-functionalized lysine 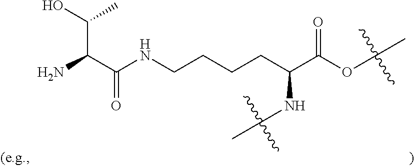 [SMILES: *N[C@@H](CCCCNC(=O)[C@@H](N)[C@@H](C)O)C(=O)O*]
or the N⁶-L-threonyl-functionalized lysine derivative

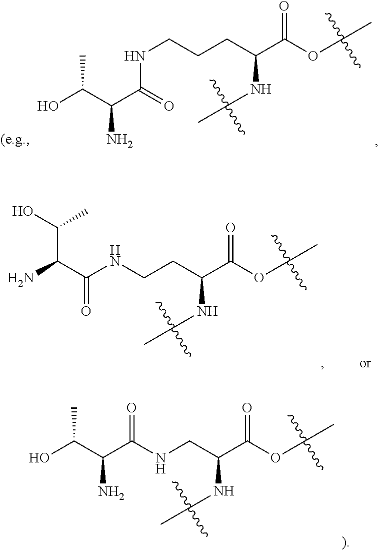 [SMILES: *N[C@@H](CCCNC(=O)[C@@H](N)[C@@H](C)O)C(=O)O* ; *N[C@@H](CCNC(=O)[C@@H](N)[C@@H](C)O)C(=O)O* ; *N[C@@H](CNC(=O)[C@@H](N)[C@@H](C)O)C(=O)O*]

4. The modified polypeptide of any one of claims 1 to 3, wherein the polypeptide comprises an amino acid sequence that is at least 90%, or 92%, or 94%, or 95% or more, identical to glucagon-like peptide 1 fragment 7-37 (GLP-1(7-37)).

5. The modified polypeptide of any one of claims 1 to 3, wherein the polypeptide comprises an amino acid sequence that is at least 90%, or 92%, or 94%, or 95% or more, identical to parathyroid hormone fragment 1-34 (PTH(1-34)), or wherein the polypeptide comprises an amino acid sequence that is at least 85%, or 90%, or 92%, or 95% or more, identical to peptide YY 3-36 (PYY(3-36)).

6. The modified polypeptide of any one of claims 1 to 3, wherein the polypeptide comprises a peptide therapeutic selected from the group consisting of therapeutic peptides listed in Table 1, modified to comprise a lysine or a derivative thereof functionalized at M with a seryl or threonyl group.

7. The modified polypeptide of any one of claims 1 to 6, wherein the N⁶-functionalized lysine or derivative thereof is incorporated into the amino acid sequence in place of a native lysine.

8. The modified polypeptide of any one of claims 1 to 6, wherein the N⁶-functionalized lysine or derivative thereof is incorporated into the amino acid sequence in place of a native amino acid other than lysine.

9. The modified polypeptide of claim 8, wherein the native amino acid is serine.

10. A bioconjugate comprising a modified polypeptide of any one of claims 1 to 9 and a functional moiety, wherein the functional moiety is conjugated to the modified polypeptide at the seryl or threonyl group on the N⁶-functionalized lysine or derivative thereof.

11. The bioconjugate of claim 10, wherein the functional moiety comprises polyethylene glycol molecule, a lipid molecule, a fluorescent molecule, a chemiluminescent molecule, a phosphorescent molecule, a radioisotope, an enzyme, an enzyme substrate, an affinity molecule, a ligand, an antigen, a hapten, an antibody, an antibody fragment, a peptide, a peptidomimetic, a protein, a chromogenic substrate, a contrast agent, an MRI contrast agent, a PET label, a phosphorescent label, or a combination thereof.

12. The bioconjugate of claim 10, wherein the functional moiety comprises a fluorescent molecule, biotin, a polyethylene glycol molecule, a lipid molecule, or a combination thereof.

13. The bioconjugate of claim 10, wherein the functional moiety comprises: 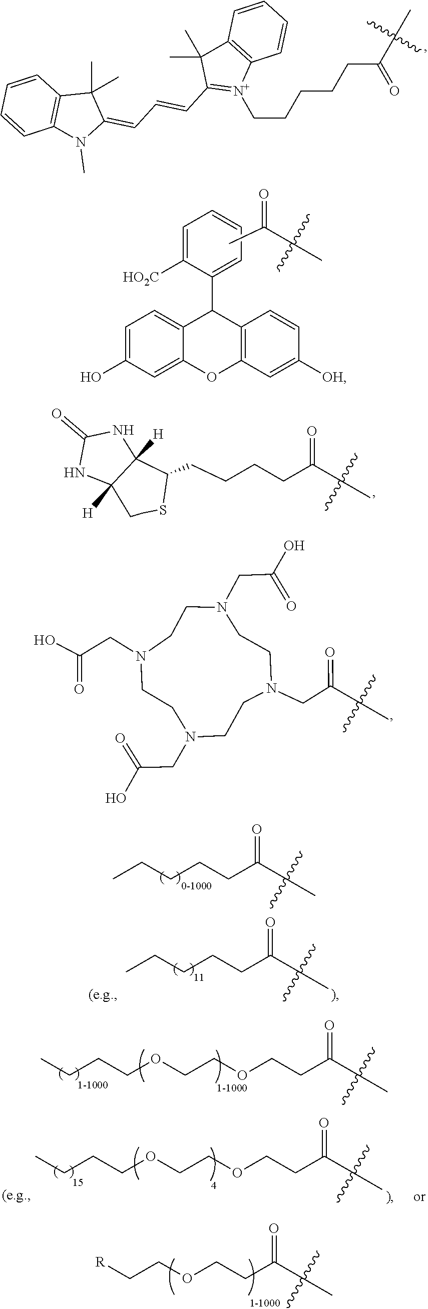
where R═NH₂, OH, or OCH₃

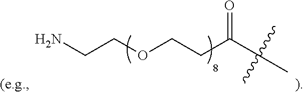

14. The bioconjugate of any one of claims 10-13, wherein the functional moiety increases biostability of the bioconjugate as compared to the biostability of the native polypeptide.

15. A pharmaceutical composition comprising one or more polypeptides and/or bioconjugates according to any one of claims 1 to 14 and a pharmaceutically acceptable carrier, solvent, adjuvant, and/or diluent.

16. A method of preparing a bioconjugate, the method comprising:
contacting a modified polypeptide comprising an amino acid sequence having a lysine or derivative thereof functionalized at M with a seryl or threonyl group with a functionalized salicylaldehyde ester, wherein the salicylaldehyde ester reacts with the seryl or threonyl group on the N⁶-functionalized lysine or derivative thereof to obtain the bioconjugate.

17. The method of claim 16, wherein the functionalized salicylaldehyde ester is of formula: 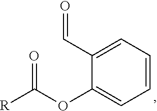
wherein R is moiety that comprises a polyethylene glycol molecule, a lipid molecule, a fluorescent molecule, a chemiluminescent molecule, a phosphorescent molecule, a radioisotope, an enzyme, an enzyme substrate, an affinity molecule, a ligand, an antigen, a hapten, an antibody, an antibody fragment, a peptide, a peptidomimetic, a protein, a chromogenic substrate, a contrast agent, an MRI contrast agent, a PET label, a phosphorescent label, or a combination thereof; or a fluorescent molecule, biotin, a polyethylene glycol molecule, a lipid molecule, or a combination thereof).

18. The method of claim 16, wherein the functionalized salicylaldehyde ester is of formula: 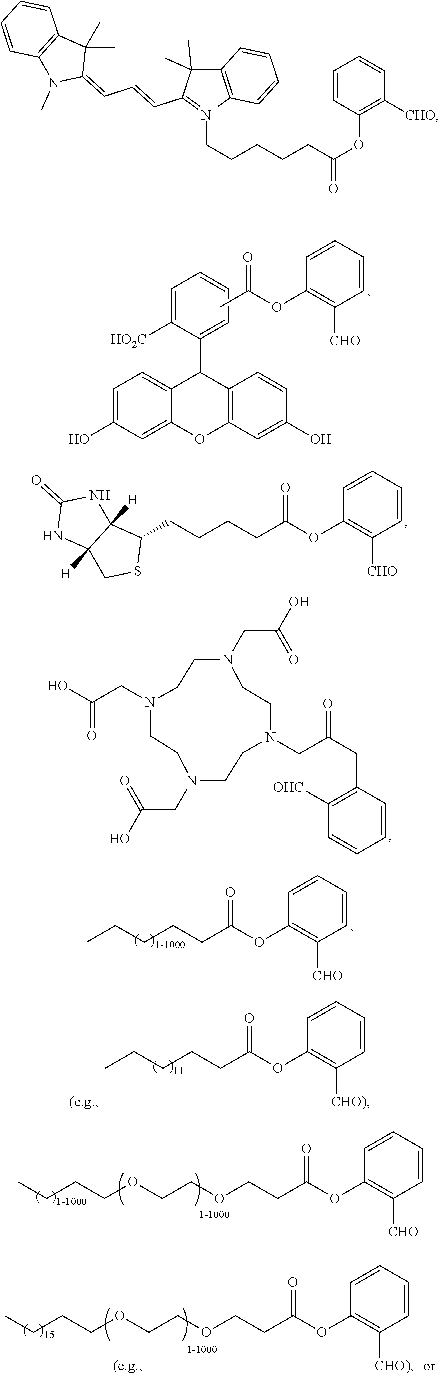 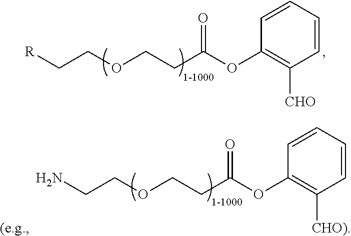
where R═NH₂, OH, or OCH₃

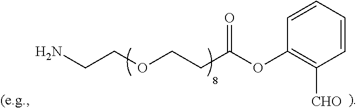

19. The method of claim 16, wherein R moiety increases biostability of the bioconjugate as compared to the biostability of the modified polypeptide.

20. The method of any of claims 16 to 19, wherein the modified polypeptide is as described in any of claims 2 to 9.

21. The method of any of claims 16 to 20, wherein the modified polypeptide is contacted with the functionalized salicylaldehyde ester in pyridine/acetic acid solution; or wherein the modified polypeptide is contacted with the functionalized salicylaldehyde ester in 1:1 v/v ratio of a pyridine/acetic acid solution.

22. The method of any of claims 16 to 21, wherein the modified polypeptide is contacted with the functionalized salicylaldehyde ester at temperature in a range of about 18° C. to 27° C. for a period of time sufficient to form a N,O-benzylidene acetal intermediate.

23. The method of claim 22, wherein the N,O-benzylidene acetal intermediate is reacted under acidic conditions for a period of time sufficient to form the bioconjugate.

24. The method of claim 23, wherein trifluoroacetic acid is used.

25. The method of any of claims 16 to 24, further comprising introducing a N⁶-seryl-lysine or derivative thereof or N⁶-threonyl-lysine or derivative thereof during the modified polypeptide synthesis.

26. The method of any of claims 16 to 24, wherein the modified polypeptide comprises a N-terminal serine or threonine, and the method further comprises protecting the N-terminal serine or threonine with a protecting group prior to contacting with the functionalized salicylaldehyde ester.

27. The method of claim 26, wherein the protecting group is selected from allyl serine and N-terminal acetylation.


SEQUENCE LISTING (6 sequences)

SEQ ID NO: 1 - protein, 31 aa, synthetic construct
HAEGTFTSDVSSYLEGQAAKEFIAWLVKGRG

SEQ ID NO: 2 - protein, 34 aa, synthetic construct
SVSEIQLMHNLGKHLNSMERVEWLRKKLQDVHNF

SEQ ID NO: 3 - protein, 34 aa, synthetic construct
IKPEAPGEDASPEELNRYYASLRHYLNLVTRQRY

SEQ ID NO: 4 - protein, 31 aa, synthetic construct
HZEGTFTSDVSSYLEGQAAUEFIAWLVRGRG

SEQ ID NO: 5 - protein, 31 aa, synthetic construct
  UNSURE 20: note = any amino acid
HZEGTFTSDVSSYLEGQAAXEFIAWLVKGRG

SEQ ID NO: 6 - protein, 31 aa, synthetic construct
  UNSURE 12: note = any amino acid
HAEGTFTSDVSXYLEGQAAKEFIAWLVKGRG